(19) United States
(12) Patent Application Publication
(10) Pub. No.: US 2022/0110575 A1
(43) Pub. Date: Apr. 14, 2022

(54) METHOD AND SYSTEM FOR IDENTIFYING FIDUCIAL FEATURES IN THE CARDIAC CYCLE AND THEIR USE IN CARDIAC MONITORING

(71) Applicant: SOTERA WIRELESS, INC., San Diego, CA (US)

(72) Inventors: Mohsen NAJI, San Diego, CA (US); Devin McCOMBIE, San Diego, CA (US)

(73) Assignee: SOTERA WIRELESS, INC., San Diego, CA (US)

(21) Appl. No.: 17/500,836

(22) Filed: Oct. 13, 2021

Related U.S. Application Data
(60) Provisional application No. 63/091,228, filed on Oct. 13, 2020.

(51) Int. Cl. A61B 5/35; A61B 5/11; A61B 5/00; A61B 5/02; A61B 5/339; G16H 50/30; G16H 40/63

(52) U.S. Cl. CPC A61B 5/35; A61B 5/1102; A61B 5/7221; G16H 40/63; A61B 5/339; G16H 50/30; A61B 5/02028

(57) ABSTRACT

A method and body-worn monitoring system for continuous fiducial point determination in SCG and ECG signals.

### CROSS-REFERENCE TO RELATED APPLICATIONS

The present application claims benefit of U.S. Provisional Application No. 63/091,228, filed Oct. 13, 2020, which is hereby incorporated by reference in its entirety including all tables, figures, and claims and from which priority is claimed.

### BACKGROUND OF THE INVENTION

Cardiac time intervals have clinical significance patient monitoring, and can provide important medical data in conditions such as mitral valve stenosis, coronary artery disease, arterial hypertension, atrial fibrillation, hypovolemia and fluid responsiveness, chronic myocardial disease and the assessment of left ventricular performance. Some of the important cardiac intervals include pre-ejection period (PEP), defined as the time period between the onset of left ventricular depolarization (typically determined by the onset of QRS complex on an electrocardiogram (ECG) and the opening of the aortic valve; left ventricular ejection time (LVET), defined as the interval between aortic valve opening and closing; total systolic time (TST), defined as the time between the Q wave and the closure of the aortic valve; and electromechanical delay (EMD), defined as the time interval between the Q wave and the closure of the mitral valve. Estimation of these cardiac intervals requires detecting the timing of the opening and closure of the aortic and mitral valves, which are not easily determined from an ECG.

ECG signals are electronically converted signals from the depolarization and repolarization of the atria and ventricle. Generally, signals are composed of a P-wave, QRS complex, and T-wave, which occurred in the depolarization of the atrium and ventricle, and the repolarization of the ventricle, respectively. The ECG can be supplemented with methods such as Doppler flow imaging, tissue Doppler Imaging (TDI), phonocardiography (PCG), impedance cardiography (ICG) and seismocardiography (SCG) for identifying the missing valve openings and closings. Each of these methods require signal analysis to identify fiducial features in the measured signals indicative of a particular event in the cardiac cycle. Signal analysis can be thought of as a combination of noise removal, fiducial point detection, and feature value acquisition and classification. The detection of accurate fiducial points is key to accurate cardiac monitoring.

### SUMMARY OF THE INVENTION

The present invention provides a method and body-worn monitoring system for continuous fiducial point determination in SCG and ECG signals. These fiducial points provide an improved system and method for monitoring pre-ejection period (PEP) and identifying clinically important changes therein (ΔPEP). The methods and systems described herein continuously derive patient-specific SCG templates by analyzing slices of SCG data. The onset of the SCG slices are determined by an ECG cardiac beat detection algorithm operating on the monitoring system. The monitoring system continuously detects robust waves in the SCG templates (region of interest, ROI) to set a reference for ΔPEP measurement. The ROI extraction does not require deterministic annotation of the physiologic events (e.g, aortic valve opening for PEP).

For individual cardiac beats, ΔPEP measurement starts with user trigger (calibration). The algorithm uses the most recent ROI prior to the calibration and a fitness function to measure ΔPEP. The ΔPEP measurement can be reset by any calibration at any time. Using ΔPEP can also improve the accuracy of continuous blood pressure (cNIBP) estimation in systems that use pulse arrival time (PAT) for cNIBP estimation.

In a first aspect, the invention relates to methods for monitoring fiduciary features in the cardiac cycle of an individual, comprising:
generating a time-dependent seismocardiogram waveform using a vibration sensor located on the thorax of the individual;
generating a corresponding time-dependent ECG waveform using an ECG sensor located on the individual;
receiving the time-dependent seismocardiogram waveform and the time-dependent ECG waveform on a processing component and executing code on the processing component,
wherein executing the code performs the following steps to process the time-dependent seismocardiogram waveform and the time-dependent ECG waveform
filtering the time-dependent seismocardiogram waveform to a frequency band between 0 Hz and 100 Hz to create a filtered seismocardiogram waveform;
creating a template, wherein the template is an average seismocardiogram waveform window calculated from at least 10 windows meeting a quality metric, by
   (i) for each QRS complex n identified in the time-dependent ECG waveform, segmenting the filtered seismocardiogram waveform in a window n, with each window being l₁ msec in length,
   (ii) determining a quality metric for window n for potential inclusion in the template,
   (iii) including window n in the template if the quality metric is acceptable,
   (iv) repeating (i)-(iii) until at least 30 windows are included in the template, and
identifying a fiducial point in the template indicative of aortic valve opening;
for each subsequent QRS complex m in the filtered seismocardiogram waveform following arrival at a template, identifying an aortic valve opening m corresponding to the QRS complex m in the filtered seismocardiogram waveform by segmenting the filtered seismocardiogram waveform in a window m with each window m being l₂ msec in length, and comparing window m to the template using a fitness function and identifying a fiducial point in window m that matches the fiducial point in the template.

The value of l₁ and l₂ may be determined based on an actual heart rate for the individual such that each beat is effectively sampled. For example, at a heart rate of 180 beats per minute (3 beats per second), a value of l₁ and l₂ of about 333 msec would capture each heartbeat. In certain embodiments, values of l₁ and l₂ are selected such that any reasonable heart rate would be sampled. The heart rate in atrial fibrillation may range from 100 to 175 beats a minute, while the normal range for a heart rate is 60 to 100 beats a minute. In preferred embodiments values of l₁ and l₂ of at least about 256 msec are selected such that it is unlikely that each heartbeat will not be sampled effectively.

In certain embodiments, each subsequent QRS complex m in the filtered seismocardiogram waveform is used to update the template according to steps (i)-(iv). In this way, the template may be continuously updated according to the latest seismocardiogram waveform data for the individual.

Suitable vibration sensors that find use in the present invention include accelerometers, gyroscopes, laser Doppler vibrometers, microwave Doppler vibrometers, and airborne ultrasound surface motion cameras. This list is not meant to be limiting.

In certain embodiments, the time-dependent seismocardiogram waveform is recorded on a dorsoventral axis. This is an axis passing through the torso from back to front. A preferred frequency band for the time-dependent seismocardiogram waveform is between about 6 Hz and about 60 Hz, which may be obtained through filtering of a broader set of recorded frequencies.

In various embodiments, the quality metric may be determined from a minimum-to-maximum amplitude (“minmax”), a normalized energy for 120 msec interval (“nE”), a variance of a derivative calculated for the segment (“nVD”), and a number of threshold crossings (“THC”) for window n. In certain embodiments, these values are calculated as follows:
MinMax(n)=max(x[n])−min(x[n]), where x[n] is the amplitude of the filtered seismocardiogram waveform in window n;

$$nE ( n ) = ( {\sum }_{n = 1}^{0.12 * Fs} {x [ n ]}^{2} - {\sum }_{n = 0.12 * Fs}^{N 1} {x [ n ]}^{2} ) / ( {\sum }_{n = 1}^{0.12 * Fs} {x [ n ]}^{2} + {\sum }_{n = N 2}^{Nb} {x [ n ]}^{2} ) ; \\ nVD ( n ) = MinMax ( n ) / ( 1 + ( {\sum }_{n = 2}^{Nb} {( x [ n ] - x [ n - 1 ] )}^{2} ) / ( Nb - 1 ) ) ; and \\ THC ( n ) = {\sum }_{n = 1}^{Nb - 1} f ( ( x [ n ] - Th ) * ( x [ n + 1 ] - Th ) ) \qquad ( 5 )$$

where f(s)=1 if s<0 otherwise f(s)=0, Th=r*Max(x[n]), x[n]=1,2, . . . ,Nwin, 0<r<1

In preferred embodiments, the template is an average seismocardiogram waveform window calculated from at least 10 windows, 20 windows, 30 windows, 40 windows, 50 windows, 60 windows, or more, meeting a desired quality metric.

Once a template is established, each fiducial point in window m that matches the fiducial point in the template can be used to derive a preejection period corresponding to each QRS complex m. This PEPm can also be used as a correction value for a pulse transit time measurement in order to derive a continuous noninvasive blood pressure value. Thus, in certain embodiments, the processing component can execute code that performs the following steps
for each aortic valve opening m and QRS complex m, calculating a preejection period (PEP)m as the time difference between the onset of QRS complex m and occurrence of aortic valve opening m; and displaying each PEPm on a display device; and
for each aortic valve opening m and QRS complex m, calculating a pulse transit time (PTT) m using PEP m, and a continuous noninvasive blood pressure (cNIBP) value m using PTT m; and displaying the cNIBP value m on the display device.

In a related aspect, the present invention provides a system for monitoring fiduciary features in the cardiac cycle of an individual according to the methods described herein. Such a system comprises:
a vibration sensor configured to position externally on the thorax of the individual and generate a time-dependent seismocardiogram waveform;
an ECG sensor configured to position externally on the individual and generate a time-dependent ECG waveform;
a processing component comprising a microprocessor and a non-volatile memory operably connected to the microprocessor, wherein the processing component is operably connected the vibration sensor and the ECG sensor to receive the time-dependent seismocardiogram waveform and the time-dependent ECG waveform and is configured to execute code stored on the processing component, wherein executing the code performs the following processing steps on the time-dependent seismocardiogram waveform and the time-dependent ECG waveform
filtering the time-dependent seismocardiogram waveform to a frequency band between 0 Hz and 100 Hz to create a filtered seismocardiogram waveform;
creating a template, wherein the template is an average seismocardiogram waveform window calculated from at least 10 windows meeting a quality metric, by
   (i) for each QRS complex n identified in the time-dependent ECG waveform, segmenting the filtered seismocardiogram waveform in a window n, with each window being l₁ msec in length, with l₁ being at least about 256 msec,
   (ii) determining a quality metric for window n for potential inclusion in the template,
   (iii) including window n in the template if the quality metric is acceptable,
   (iv) repeating (i)-(iii) until at least 30 windows are included in the template;
identifying a fiducial point in the template indicative of aortic valve opening; and
for each subsequent QRS complex m in the filtered seismocardiogram waveform, identifying an aortic valve opening m corresponding to the QRS complex m in the filtered seismocardiogram waveform by segmenting the filtered seismocardiogram waveform in a window m with each window m being l₂ msec in length, comparing window m to the template using a fitness function and identifying a fiducial point in window m that matches the fiducial point in the template.

### BRIEF DESCRIPTION OF THE FIGURES

FIG. 1 depicts an example of accepted and rejected beats for SCG template.

FIGS. 2a and 2b depict the effect of template size on capturing different cardiac events.

FIG. 3 depicts an example of ΔPEP extracted from ECG and SCG using a template region of interest (left inset) calculated before the user trigger at time 0. Top middle and right insets show examples of fiducial point detection using the template and applied fitness function.

FIG. 4 depicts an example of ΔPEP reset to zero after the user's second calibration trigger.

FIG. 5 depicts an example of improved cNIBP-MAP estimation (bottom panel) with PEP correction. Middle panel shows PAT and PEP-corrected PAT. Top panel shows the applied ΔPEP for cNIBP correction.

FIG. 6 depicts an example of estimated LVET by processing of SCG templates.

FIG. 7 depicts the effect of number of beats in template on template quality, PEP variance, and template availability before user's trigger.

### DETAILED DESCRIPTION OF THE INVENTION

### System Overview

For purposes of the present application, the following abbreviations apply:

| Terminology | Definition |
|---|---|
| ECG | Electrocardiogram |
| SCG | Seismocardiogram |
| PPG | Photoplethysmogram |
| PEP | Pre-ejection Period |
| ΔPEP | Changes in PEP |
| LVET | Left ventricle ejection time |
| NIBP | Non-invasive blood pressure |
| cNIBP | Continuous Non-invasive blood pressure |
| MAP | Mean arterial pressure |
| ASYS | Asystole |
| AFIB or AF | Atrial Fibrillation |
| VFIB or VF | Ventricular Fibrillation |
| VTACH | Ventricular Tachycardia |
| LTA + AF | Life Threatening Arrhythmias |
| | plus Atrial Fibrillation |
| RR | Interval between successive QRS |
| | complexes |
| HR | Heart Rate |
| PPV | Positive Predictive Value |
| PI | Pulse Interval |
| ACC | Accelerometer |
| PWD | Patient Worn Device |
| RVD | Remote Viewing Device |

For purposes of example only, the present invention is described in terms of using the ViSi Mobile® vital sign monitoring system (Sotera Wireless, Inc.). The ViSi Mobile system is a body-worn vital sign monitor that continuously measures heart rate, SpO2, respiration rate, pulse rate, blood pressure, and skin temperature. The body worn monitor is comprised of a wrist device and a cable, which includes an upper arm module and a chest module as shown in U.S. Pat. No. 8,321,004. The wrist device, upper arm module, and chest module each contain a three-axis accelerometer. In addition to the more traditional vital signs, the three accelerometers in the monitor capture data that can be used to estimate a patient's posture, the time spent in a specific posture, detect when a patient has fallen, and determine when the patient is walking.

### Seismocardiography

The Seismocardiogram (SCG) provides an ideal non-invasive way to measure body vibration which are induced by the operation of heart valves in a body worn monitor. SCG captures the chest acceleration induced by the motion of myocardium recorded using an accelerometer commonly mounted on the lower part of the sternum. SCG signals are the cardiac vibrations measured noninvasively at the chest surface. The SCG signals have multiple spectral peaks at 9.20 ±0.48, 25.84 ±0.77, 50.71 ±1.83 Hz (mean±SEM) (The higher frequency component (>20 Hz) of the SCG has a close morphological similarity to phonocardiogram (PCG)). As early as 1957, SCG was recorded under the name of precordial ballistocardiogram and was used in the early 1960s for monitoring heart rate variability. Afterward, in the late 1980s, SCG was introduced as a technology for monitoring cardiac function. In a study conducted by Crow et al., the fiducial points of the SCG, labeled as MC, AO, AC, and MO were found to correspond to mitral valve closure, aortic valve opening, aortic valve closure and mitral valve opening, respectively, and validated against echocardiography images.

### Identifying Fiducial Features

The accurate estimation of PEP depends on detection of AO wave in SCG. Simultaneous SCG and ultrasound images have shown that timing of several waves in SCG during systole phase of cardiac cycle had significant positive correlation with AO timing in ultrasound images. Because the following is described in terms of changes in PEP, the approach is to identify the most robust wave (region of interest, ROI) in SCG beat templates and calculate the changes in timing of that selected wave as ΔPEP. That is, there is no need for deterministic annotation of SCG waves to calculate ΔPEP. Depending on the template window size, changes in timing of AO and aortic closure (AC) can be estimated.

The method continuously extracts segments from filtered SCG and evaluates the SCG segments by several feature extraction techniques that were developed based on a dataset of annotated segments. This beat evaluation, continuously generates information about beat quality and minimizes the chance of including noisy beats in the SCG template. The SCG template is updated every Nt acceptable SCG segments. Features are extracted to find the most robust region of interest (wave), in the SCG template, for tracking changes in PEP (ΔPEP). After the user trigger (e.g., blood pressure calibration), for every heart beat a fitness function evaluates local extrema of SCG segments against the template region of interest to calculate ΔPEP. For the subsequent user triggers, ROI will be updated and ΔPEP resets to zero.

### Filtering

A linear phase band-pass filter is designed to filter 6-60 Hz components of the dorsoventral (back-to-front) axis of SCG data. The filtered SCG data are buffered to overcome filtering delays and processing time for detection of the QRS complex in ECG.

PT and Gravity Cliff Detector to Identify Ventricular Depolarization in ECG, leads I, II, and III

An appropriate gravity cliff detector is described in PCT/US2019/052706, which is hereby incorporated by reference in its entirety. For every ECG lead, the Pan-Tompkins (PT) algorithm produces a pulse for each QRS complex. The gravity cliff detector (GCD) algorithm fuses the PT waveforms based on ECG quality. The GCD simulates constant negative acceleration on a particle that is moving with time along the signal. The magnitude of the fused signal is interpreted as a height value. As the particle falls off the top of a peak in the signal, it accelerates towards the signal baseline and its velocity increases analogous to a freefall. While in this freefall state if the velocity exceeds a threshold then a cliff is detected at the time and amplitude value of the signal at the start of the free fall period. For every detected cliff, the QRS complex is detected as the midpoint of the fused signal exceeding 80% of the cliff height.

### QRS Complex Fiducial Point to Window SCG Waveform

For every detected QRS complex fiducial point in ECG, slices of Nb samples from the filtered SCG are taken (“windows”). The algorithm uses the SCG beat segments to update the SCG template and to calculate changes in PEP for every beat. Choice of Nb determines the maximum heart rate (HR) at which ΔPEP can be calculated:

$${HR}_{max} ( beats per minute ) = 60 / Nb * Fs ) , \qquad ( 1 )$$

where Fs is sampling rate (Hz).

### Evaluate SCG Signal Quality Using a Set of Features and a Simple Classifier

For every SCG segment x[n], n=1,2, . . . ,Nb, (n=1 occurs at the QRS complex sequence number) a set of features are extracted:

1) minimum-to-maximum amplitude:

$$MinMax = max ( x [ n ] ) - min ( x [ n ] ) \qquad ( 2 )$$

2) normalized energy for 120 msec interval:

$$nE = ( {\sum }_{n = 1}^{0.12 * Fs} {x [ n ]}^{2} - {\sum }_{n = 0.12 * Fs}^{N 1} {x [ n ]}^{2} ) / ( {\sum }_{n = 1}^{0.12 * Fs} {x [ n ]}^{2} + {\sum }_{n = N 2}^{Nb} {x [ n ]}^{2} ) \qquad ( 3 )$$

where N1<Nb, N2<Nb.

3) normalized variance of derivative of SCG segment:

$$nVD = MinMax / ( 1 + ( {\sum }_{n = 2}^{Nb} {( x [ n ] - x [ n - 1 ] )}^{2} ) / ( Nb - 1 ) ) \qquad ( 4 )$$

4) number of threshold crossings:

$$THC = {\sum }_{n = 1}^{Nb - 1} f ( ( x [ n ] - Th ) * ( x [ n + 1 ] - Th ) ) \qquad ( 5 )$$

where f(s)=1 if s<0 otherwise f(s)=0, Th=r*Max(x[n]), x[n]=1,2, . . . ,Nwin, 0<r<1

A set of boundaries for the mentioned features, and a binary classifier are used to evaluate SCG segments.

For X=[MinMax, nE, nVD, THC] as features of x[n] and f_{good_beat}(.) as a binary classifier:

f_{good_beat} (X)=1 if x[n] is good for template, otherwise f_{good_beat} (X)=0 .

### Create an SCG Template When Monitoring Starts

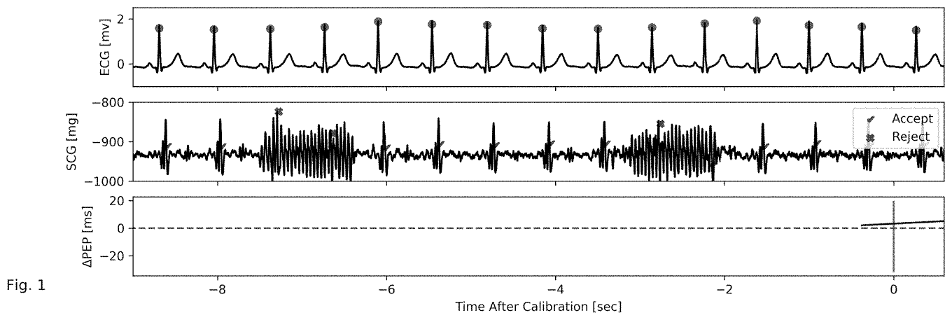

An SCG template is continuously updated by averaging Nt (between 10 and 60 beats) acceptable beats (FIG. 1). We performed an analysis on 58472 patient data to find optimum Nt. We tested the effect of 10, 35, and 60 beats in the template on the following outcomes: 1) PEP variance. Higher PEP variance implies more uncertainty in SCG fiducial point selection. 2) A template quality metric: summation of absolute value of template peaks and valleys normalize by template maximum value. Lower values for this quality metric relate to higher quality templates. 3) Availability of a template before user input. Using 35 beats for creating the template provides a reasonable tradeoff between having a template before user input and quality of the template and calculated PEP.

### Extract a Region of Interest

Initialization: Template T[n] = 0, n=1,2, ..., Nb; beat_counter = 0.

For every new beat x[n]:
If beat_counter<=Nt

if fgood_beat (X) = 1:

beat_counter = beat_counter + 1;
T[n] = T[n] + x[n]/Nt, n=1,2,..., Nb
If beat_counter = Nt

Calculate SCG Template region of interest ROI

([ROI_{Lower}_{_{—}}_{boundary}, ROI_{AO}, ROI_{Upper}_{_{—}}_{boundary}] = f_{ROI}(T[n]) )

Reset template: T[n] = 0, n=1,2, ..., Nb; beat_counter = 0.

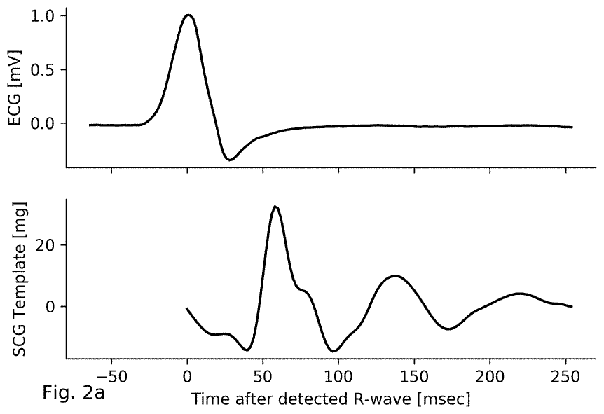

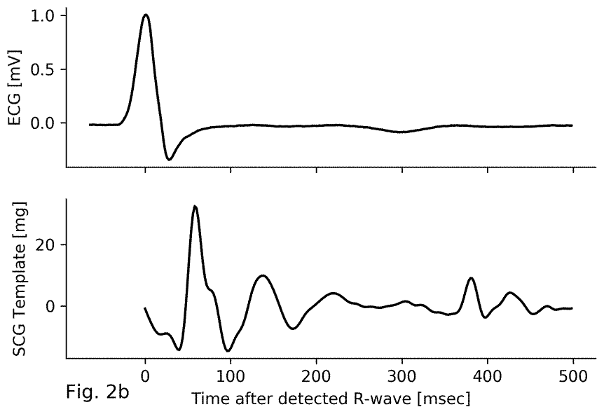

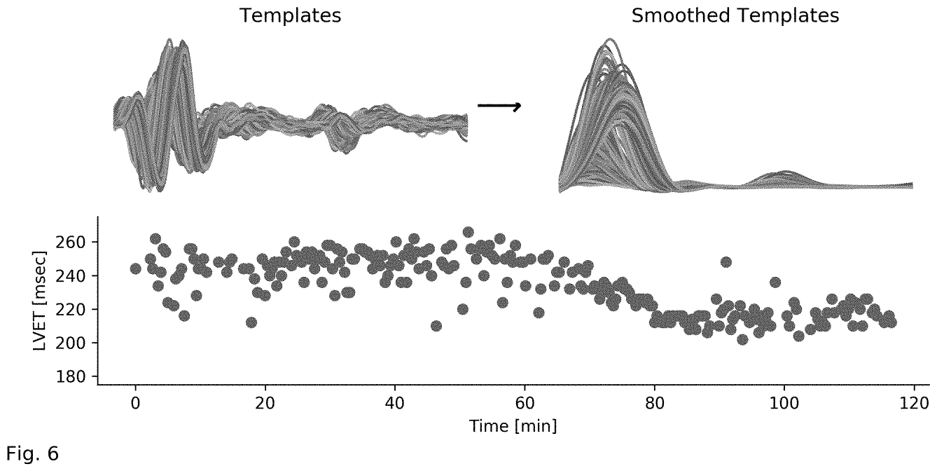

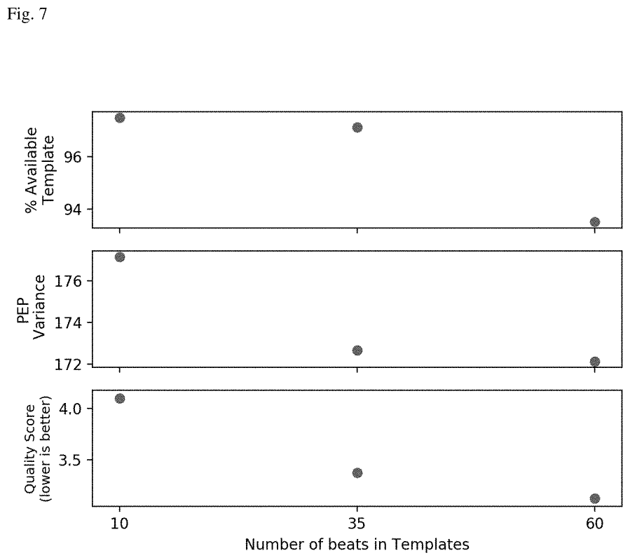

Template size (Nb) can be chosen in a way to capture systole events, including aortic valve opening (AO), for ΔPEP detection (FIG. 2a) or can be chosen longer to capture both systole and diastole events including aortic valve closure (AC) (FIG. 2b) to measure the left ventricle ejection time (LVET, calculated as AC−AO timing). In the latter case, a smooth version of template can be derived by low pass filtering the square of first derivative of template as

$$sT [ n ] = LPF ( {( T [ n ] - T [ n - Δ n ] )}^{2} ) \qquad ( 6 )$$

where, LPF(.) is a low-pass filter. After calculation of sT[n], LVET can be estimated as the timing between major peaks of sT[n] (FIG. 6) spaced at least LVET_ min samples.

### Identify Fiducial Point for Aortic Valve Opening in SCG Template

Every extremum point in the template is scored based on amplitude, sharpness, and distance to the most probable SCG event (e.g, AO) timings (relative to QRS complex in ECG) in an annotated dataset.

For extremum time n=i in T[n], n=2,3, . . . ,Nb-1:

$$Score ( T [ i ] ) = {f}_{score} ( T [ i ] , \vert 2 * T [ i ] - T [ i - 1 ] - T [ i + 1 ] \vert , \vert i - {n}_{most \_ probable \_ AO} \vert )$$

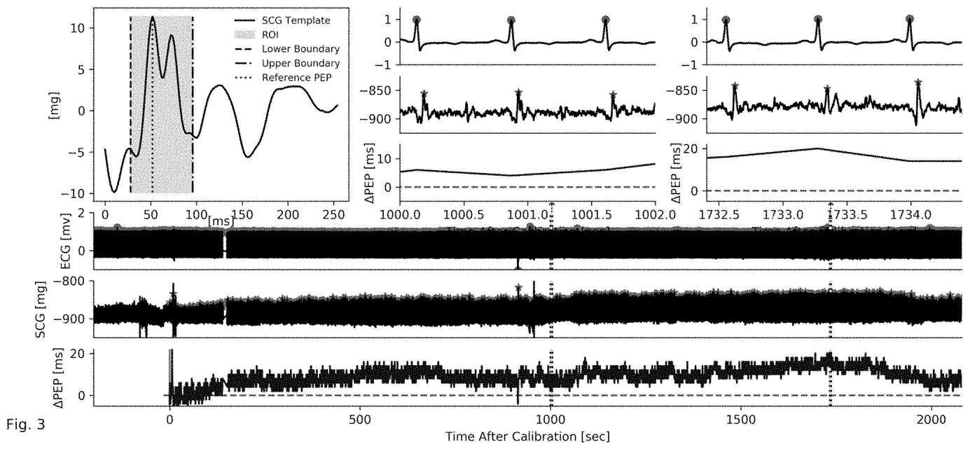

The extremum with the highest score is considered as the reference for template region of interest (ROI_{AO}). Lower (ROI_{lower_boundary}) and upper (ROI_{upper_boundary}) boundaries of the ROI are identified from the extrema before and after the reference point (FIG. 3).

### Use the Waveform Template and Template Fiducial Point to Identify Fiducial Points for Subsequent Cardiac Cycles

After a user trigger (e.g, calibration) the most recent template ROI is set as reference to track PEP changes. For each cardiac cycle after the trigger, a fitness function uses ROI parameters to evaluate the timing and amplitude of SCG segment (x[n]) extrema in the ROI window and calculates the change in PEP (FIG. 3).

For extremum time n=j in x[n], ROI_{lower_boundary}<n<ROI_{upper_boundary}:

$$Fitness ( x [ j ] ) = {f}_{fitness} ( j , x [ j ] , min ( x [ n ] ) , {ROI}_{AO} )$$

For sample n=k in x[n] with the maximum Fitness(x[k]):

$$P E P = k * 1000 / Fs ( in msec )$$

$$Δ P E P = ( k - {ROI}_{AO} ) * 1000 / Fs ( in msec )$$

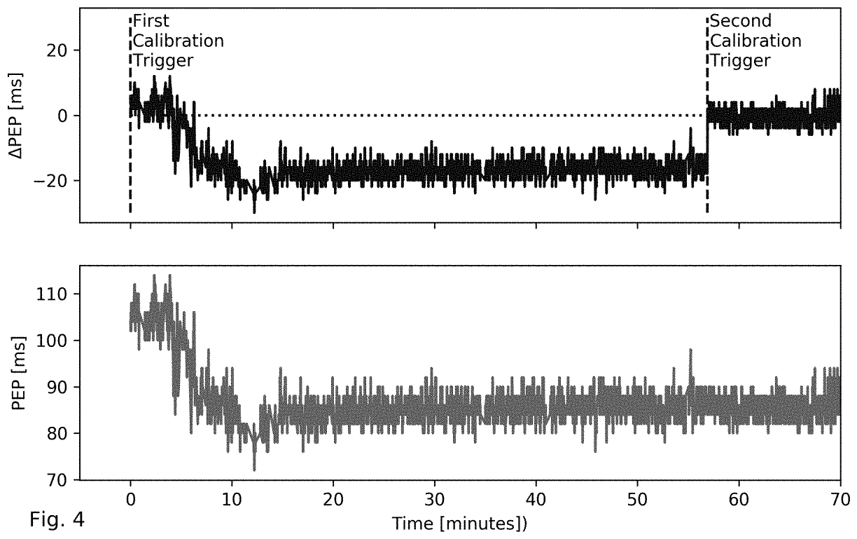

With every user's calibration trigger, template ROI updates and ΔPEP resets to zero (FIG. 4).

### Monitoring System

The aortic valve fiducial point determined from the described method can be used to calculate changes in PEP when used with the ECG and can be used to compensate for changes in PEP in continuous non-invasive blood pressure (CNIBP) when combined with ECG and PPG.

A body worn system can be utilized with ECG (lead I, lead II, and lead III), PPG (e.g., measured at the base of one of the digits), SCG (attached to the torso, and preferably the sternum). Simultaneous recording and processing of ECG and SCG as well as a user trigger (calibration) are required for calculation of changes in PEP (ΔPEP). A sampling rate of >=500 Hz is recommended to achieve better temporal resolution for PEP change estimation.

Correction of Continuous Non-Invasive Blood Pressure (cNIBP)

The ViSi Mobile® system (Sotera Wireless) measures the continuous non-invasive blood pressure (cNIBP) based on pulse arrival time (PAT). This yields individual blood pressure values (systolic or “SYS, diastolic or “DIA’, and mean arterial or “MAP). PAT can be measured on a beat-to-beat basis as the time difference between the onset of the photoplethysmogram (PPG) at the base of the thumb (or index finger) and the peak of the QRS complex in the ECG waveform. The wrist module of the ViSi Mobile System records PPG signals.

The measured time difference is the sum of the true vascular transit time (VTT), i.e. the time interval required for the pulse to propagate from the heart to the PPG sensor location, and the pre-ejection period (PEP):

$$PAT = V T T + P E P \qquad ( 7 )$$

PAT typically relates inversely to blood pressure, i.e., a decrease in PAT indicates an increase in blood pressure. Values for systolic, diastolic, and mean arterial pressure are determined for every periodically aggregated PAT value (PATnum) using the following formulas:

$$\begin{matrix}MAP = K ( 1 / P A {T}_{num} - 1 / P A {T}_{cal} + {MAP}_{cal} & ( 8 ) \\ SYS = {R}_{SYS} . MAP & ( 9 ) \\ DIA = {R}_{DIA} . MAP & ( 10 )\end{matrix}$$

where calibration parameters K, MAP_{cal}, R_{SYS}, and R_{DIA} are identified using the NIBP module of the ViSi Mobile System and PATcal represents an aggregate PAT measured at the time of the NIBP inflation.

According to (7), changes in PAT (ΔPAT) can be due to ΔVTT and ΔPEP:

$$Δ P A T = Δ V T T + Δ P E P \qquad ( 11 )$$

Every blood pressure calibration in the ViSi Mobile System sends a message from the wrist module to the chest module and starts or restarts the ΔPEP measurement.

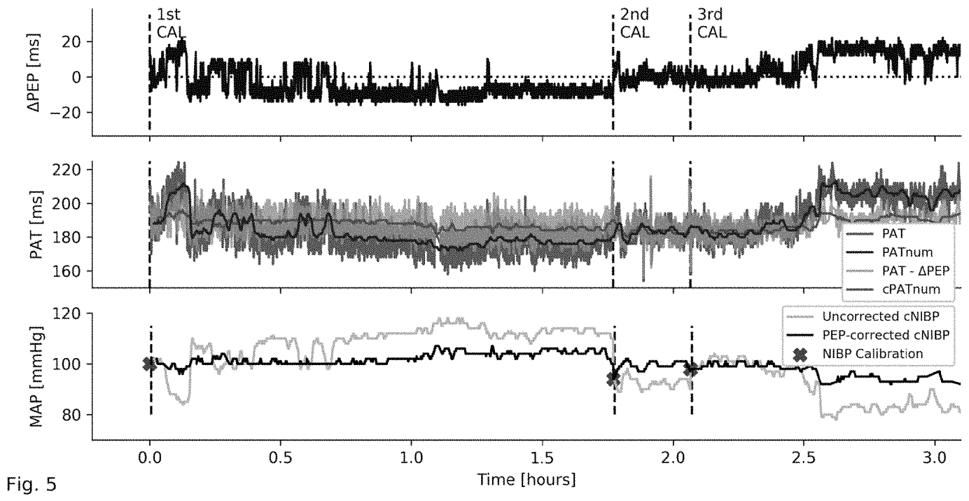

For correlated changes in ΔPEP and ΔPAT, the corrected PAT (cPAT) is calculated as (FIG. 5):

$$cPAT = P A T - Δ P E P \qquad ( 12 )$$

where ΔPEP (FIG. 5) is extracted from the described algorithm [in sectionX] for every cardiac beat. The described SCG beat quality metrics may be used for conditional PAT correction.

Other corrections (e.g, arm height change correction or torso posture change correction) may be applied to PAT as described in, for example, U.S. Pat. Nos. 8,321,004; 9,364,158; 10,342,438; 10,213,159; 9,901,261; 8,602,997; and U.S. Pat. No. 10,004,409, each of which is hereby incorporated by reference in its entirety.

Aggregated cPAT (cPATₙᵤₘ) values (FIG. 5 middle) may be used to update the cNIBP MAP estimation equation (FIG. 5 bottom):

$$MAP = K ( 1 / cP A {T}_{num} - 1 / P A {T}_{cal} ) + {MAP}_{cal} \qquad ( 13 )$$

PAT, PTT and Blood Pressure

It is also an object of the present invention to provide methods and systems for continuous noninvasive measurement of vital signs such as blood pressure (cNIBP) based on PAT, which features a number of improvements over conventional PAT measurements. Pulse transit time (PTT) is the time it takes for the pressure or flow wave to propagate between two arterial sites, and has been shown to correlate fairly well with acute changes in BP over a wide physiological BP range. PTT estimated as the time delay between noninvasive proximal and distal arterial waveforms could therefore permit convenient tracking of BP changes. Indeed, noninvasive PTT estimates are being widely pursued at present for cuff-less BP monitoring.

The most popular noninvasive PTT estimate has been the time delay between ECG and photoplethysmography (PPG) waveforms, referred to as pulse arrival time (PAT). However, the major concern is that PAT not only includes PTT but also the pre-ejection period (PEP), which varies with cardiac electrical and mechanical properties.

The invention uses a body-worn monitor that recursively determines an estimated PEP for use in correcting PAT measurements by detecting low frequency vibrations created during a cardiac cycle, and using a state estimator algorithm to identify signals indicative of aortic valve opening in those measured vibrations. An uncorrected PAT is determined conventionally from the onset of the cardiac cycle and the time at which the corresponding pressure pulse is identified using photoplethysmography. PEP is then determined for each cardiac cycle on a beat-to-beat basis based on the difference between onset of the cardiac cycle and the currently estimated time of aortic valve opening according to the methods described herein. Using these values, a cNIBP measurement is obtained following correction of the PAT for PEP. Various vital signs obtained from such a body-worn system of sensors may be transmitted to a remote monitor, such as a tablet PC, workstation at a nursing station, personal digital assistant (PDA), or cellular telephone.

### Sensor Configurations

A cNIBP monitor can comprise a torso-worn ECG/accelerometer module, a wrist transceiver/processing unit, a pulse oximetry module and NIBP module which determines an oscillometric blood pressure measurement. These device components are capable of measuring four different physiologic signals; an ECG, a PPG, an SCG, and a brachial artery pressure signal that provides an oscillometric blood pressure measurement (NIBP).

The exemplified system comprises an ECG/accelerometer sensor module that includes a housing enclosing (i) an ECG circuit operably connected to a transceiver within the housing that transmits ECG waveforms (e.g., using cabling or by wireless connection) to a corresponding transceiver housed within a processing apparatus 104; and (ii) an accelerometer (e.g., ADXL-345 or LSM330D) also operably connected to the transceiver within the housing that transmits accelerometer (SCG) waveforms to a corresponding transceiver housed within a processing apparatus. ECG/accelerometer sensor module is positioned on the patient's skin at the sternum. While the ECG sensor module and the accelerometer module may be provided separately, it is advantageous for ease of use that a single housing encloses both sensor modules. Similarly, while the processing apparatus is described herein as a single body-worn processor unit, the methods and code described herein may be performed by a plurality of processors which may be housed at different locations, each of which contributes to the processing power of the system, and which are collectively therefore referred to as “the processing apparatus.” By way of example only, a processing unit may be provided at the bedside or provided in a body-worn client/remote server processor format.

In order to achieve a sufficient signal-to-noise ratio for the SCG signal the ECG/accelerometer module should be mechanically coupled to the patient's skin. The housing of the ECG/accelerometer module is secured against the patient's skin using a double-sided adhesive substance applied directly between the housing and the skin or by snapping it into a rigid fixture that is adhered to the skin. The housing should be attached at the sternum of the patient, optimally the lower sternum just above the xiphoid process. The microprocessor component of transceiver/processing apparatus applies algorithms as described below in order to collectively process ECG waveforms along with SCG waveforms to generate an improved PAT measurement.

The ECG circuit within the ECG/accelerometer module features a single circuit (e.g. an ASIC) that collects electrical signals from a series of body-worn electrodes and coverts these signals into a digital ECG waveform. Such a circuit connects to the wrist-worn transceiver through a digital, packet-based serial interface (e.g. an interface based on a “controller area network”, or “CAN”, system). Such a system can include a master clock houses in the processor module which communicates a timing packet to processors in each remote module in order to synchronize timing for the various time-dependent waveforms. Preferably, the time-dependent waveforms are synchronized such that there is a maximum 40-microsecond timing error in the synchrony between the waveforms.

The chest-worn ECG/accelerometer module connects through cables to conventional ECG electrodes located, respectively, in the upper right-hand, upper left-hand, and lower left-hand portions of the patient's thorax. Three electrodes (two detecting positive and negative signals, and one serving as a ground) are typically required to detect the necessary signals to generate an ECG waveform with an adequate signal-to-noise ratio. RED DOT™ electrodes marketed by 3M (3M Center, St. Paul, Minn. 55144-1000) are suitable for this purpose. During a measurement, the ECG electrodes measure analog signals that pass to circuits within the ECG/accelerometer module. There, ECG waveforms are generated, digitized (typically with 12-24-bit resolution and a sampling rate between 250-500 Hz), and formulated in individual packets so they can be transmitted to the wrist-worn transceiver/processing apparatus for processing.

The individual packets described above may be preferably transmitted according to the packet-based serial protocol. Use of this protocol with a wired or wireless connection between the ECG/accelerometer module and wrist-worn transceiver/processing apparatus 104 provides packets in which all timing related information between the packets is preserved such that the waveforms generated by the ECG and accelerometer may be synchronized (optionally with PPG waveforms) by the wrist-worn transceiver/processing apparatus. The protocol also permits the data corresponding to waveforms generated by the ECG and accelerometer to be segregated although transmitted by a single transceiver, as each packet can contain information designating the sensor from which the data originates.

The optical sensor detects optical radiation modulated by the heartbeat-induced pressure wave, which is further processed with a second amplifier/filter circuit within the transceiver/processing apparatus. This results in the PPG waveform, which, as described above, includes a series of pulses, each corresponding to an individual heartbeat. The depicted thumb-worn optical sensor is operably connected (wirelessly or through a cable to the wrist-worn transceiver/processing apparatus to measure and transmit PPG waveforms that, when combined with the ECG waveform, can be used to generate cNIBP measurements. This yields individual blood pressure values (systolic or “SYS”, diastolic or “DIA”, and mean arterial or “MAP”). The optical sensor additionally measures a PPG waveform that can be processed to determine SpO2 values, as described in detail in the following patent application, the contents of which are incorporated herein by reference: BODY-WORN PULSE OXIMETER, U.S. Ser. No. 12/559,379, filed Sep. 14, 2009.

In addition to the accelerometer located on the sternum within housing, the system comprises two other; one positioned on the wrist within the wrist-worn transceiver/processing apparatus and the other on the upper arm of the same arm. Each measure three unique signals, each corresponding to the x, y, and z-axes of the body portion to which the accelerometer attaches. These signals are then processed by the wrist-worn transceiver/processing apparatus 104 with a series of algorithms, some of which are described in U.S. Pat. Nos. 8,321,004; 9,364,158; 10,342,438; 10,213,159; 9,901,261; 8,602,997; and U.S. Pat. No. 10,004,409, the contents of which are incorporated herein by reference: to determine motion, posture, arm height, and activity level.

Finally, the system further comprises a pneumatic cuff-based module that communicates with the wrist-worn transceiver/processing apparatus in order to obtain oscillometric NIBP measurements. The cuff module features a pneumatic system that includes a pump, valve, pressure fittings, pressure sensor, analog-to-digital converter, microcontroller, transceiver, and rechargeable Li:ion battery. During an indexing measurement, the pneumatic system inflates a disposable cuff and performs two measurements: 1) an inflation-based measurement of oscillometry to determine values for SYSINDEX, DIAINDEX, and MAPINDEX; and 2) a patient-specific slope describing the relationship between PTT and MAP. These measurements are described in detail in U.S. Pat. No. 8,419,649, the contents of which have been previously incorporated herein by reference. Pressure waveforms are transmitted by the transceiver to the wrist-worn transceiver/processing apparatus (wirelessly or through cable) through a digital, serial interface, and preferably as packets according to the packet-based serial protocol.

The following are preferred embodiments of the invention.

1. A method of monitoring fiduciary features in the cardiac cycle of an individual, comprising:
generating a time-dependent seismocardiogram waveform using a vibration sensor located on the thorax of the individual;
generating a corresponding time-dependent ECG waveform using an ECG sensor located on the individual; and
receiving the time-dependent seismocardiogram waveform and the time-dependent ECG waveform on a processing component and executing code on the processing component, wherein executing the code performs the following steps to process the time-dependent seismocardiogram waveform and the time-dependent ECG waveform:
filtering the time-dependent seismocardiogram waveform to a frequency band between 0 Hz and 100 Hz to create a filtered seismocardiogram waveform;
creating a template, wherein the template is an average seismocardiogram waveform window calculated from at least 10 windows meeting a quality metric, by
(i) for each QRS complex n identified in the time-dependent ECG waveform, segmenting the filtered seismocardiogram waveform in a window n, with each window being li msec in length,
(ii) determining a quality metric for window n for potential inclusion in the template,
(iii) including window n in the template if the quality metric is acceptable, and
(iv) repeating (i)-(iii) until at least 30 windows are included in the template;
identifying a fiducial point in the template indicative of aortic valve opening; and
for each subsequent QRS complex m in the filtered seismocardiogram waveform, identifying an aortic valve opening m corresponding to the QRS complex m in the filtered seismocardiogram waveform by segmenting the filtered seismocardiogram waveform in a window m with each window m being l₂ msec in length, and comparing window m to the template using a fitness function to identify a fiducial point in window m that matches the fiducial point in the template.
2. A method according to embodiment 1, wherein each subsequent QRS complex m in the filtered seismocardiogram waveform is used to update the template according to steps (i)-(iv).
3. A method according to embodiment 1 or 2, wherein the vibration sensor is selected from the group consisting of an accelerometer, a gyroscope, a laser Doppler vibrometer, a microwave Doppler vibrometer, and an airborne ultrasound surface motion camera.
4. A method according to one of embodiments 1-3, wherein the time-dependent seismocardiogram waveform is recorded on a dorsoventral axis.
5. A method according to one of embodiments 1-4, wherein the frequency band is between about 6 Hz and about 60 Hz.
6. A method according to one of embodiments 1-5, wherein l₁ and l₂ are each at least about 256 msec.
7. A method according to one of embodiments 1-6, wherein the quality metric is determined from a minimum-to-maximum amplitude (“minmax”), a normalized energy for 120 msec interval (“nE”), a variance of a derivative calculated for the segment (“nVD”), and a number of threshold crossings (“THC”) for window n.
8. A method according to embodiment 7, wherein
MinMax(n)=max(x[n])−min(x[n]), where x[n] is the amplitude of the filtered seismocardiogram waveform in window n;

$$nE ( n ) = ( {\sum }_{n = 1}^{0.12 * Fs} {x [ n ]}^{2} - {\sum }_{n = 0.12 * Fs}^{N 1} {x [ n ]}^{2} ) / ( {\sum }_{n = 1}^{0.12 * Fs} {x [ n ]}^{2} + {\sum }_{n = N 2}^{Nb} {x [ n ]}^{2} ) ; \\ nVD ( n ) = MinMax ( n ) / ( 1 + ( {\sum }_{n = 2}^{Nb} {( x [ n ] - x [ n - 1 ] )}^{2} ) / ( Nb - 1 ) ) ; \\ and \\ THC ( n ) = {\sum }_{n = 1}^{Nb - 1} f ( ( x [ n ] - Th ) * ( x [ n + 1 ] - Th ) ) \qquad ( 5 )$$

where f(s)=1 if s<0 otherwise f(s)=0, Th=r*Max(x[n]), x[n]=1,2, . . . ,Nwin, 0<r<1
   9. A method according to one of embodiments 1-8, wherein the template is an average seismocardiogram waveform window calculated from at least 20 windows meeting a quality metric.
   10. A method according to one of embodiments 1-8, wherein the template is an average seismocardiogram waveform window calculated from at least 30 windows meeting a quality metric.
   11. A method according to one of embodiments 1-8, wherein the template is an average seismocardiogram waveform window calculated from at least 40 windows meeting a quality metric.
   12. A method according to one of embodiments 1-8, wherein the template is an average seismocardiogram waveform window calculated from at least 60 windows meeting a quality metric.
   13. A method according to one of embodiments 1-12, wherein executing the code further performs the following steps

for each aortic valve opening m and QRS complex m, calculating a preejection period (PEP) m as the time difference between the onset of QRS complex m and occurrence of aortic valve opening m; and

displaying each PEPm on a display device.
14. A method according to embodiment 13, wherein executing the code further performs the following steps

for each aortic valve opening m and QRS complex m, calculating a pulse transit time (PTT) m using PEP m, and a continuous noninvasive blood pressure (cNIBP) value m using PTT m; and

displaying the cNIBP value m on the display device.
15. A system for monitoring fiduciary features in the cardiac cycle of an individual, comprising:
a vibration sensor configured to position externally on the thorax of the individual and generate a time-dependent seismocardiogram waveform;
an ECG sensor configured to position externally on the individual and generate a time-dependent ECG waveform; and
a processing component comprising a microprocessor and a non-volatile memory operably connected to the microprocessor, wherein the processing component is operably connected the vibration sensor and the ECG sensor to receive the time-dependent seismocardiogram waveform and the time-dependent ECG waveform and is configured to execute code stored on the processing component, wherein executing the code performs the following processing steps on the time-dependent seismocardiogram waveform and the time-dependent ECG waveform
filtering the time-dependent seismocardiogram waveform to a frequency band between 0 Hz and 100 Hz to create a filtered seismocardiogram waveform;
creating a template, wherein the template is an average seismocardiogram waveform window calculated from at least 10 windows meeting a quality metric, by
(i) for each QRS complex n identified in the time-dependent ECG waveform, segmenting the filtered seismocardiogram waveform in a window n, with each window being li msec in length, with l₁ being at least about 256 msec,
(ii) determining a quality metric for window n for potential inclusion in the template,
(iii) including window n in the template if the quality metric is acceptable,
(iv) repeating (i)-(iii) until at least 30 windows are included in the template, and
identifying a fiducial point in the template indicative of aortic valve opening; and
for each subsequent QRS complex m in the filtered seismocardiogram waveform, identifying an aortic valve opening m corresponding to the QRS complex m in the filtered seismocardiogram waveform by segmenting the filtered seismocardiogram waveform in a window m with each window m being l₂ msec in length, with l₂ being at least about 256 msec, and comparing window m to the template using a fitness function and identifying a fiducial point in window m that matches the fiducial point in the template.
16. A system according to embodiment 15, wherein each subsequent QRS complex m in the filtered seismocardiogram waveform is used to update the template according to steps (i)-(iv).
17. A system according to embodiment 15 or 16, wherein the vibration sensor is selected from the group consisting of an accelerometer, a gyroscope, a laser Doppler vibrometer, a microwave Doppler vibrometer, and an airborne ultrasound surface motion camera.
18. A system according to one of embodiments 15-17, wherein the time-dependent seismocardiogram waveform is recorded on a dorsoventral axis.
19. A system according to one of embodiments 15-18, wherein the frequency band is between about 6 Hz and about 60 Hz.
20. A method according to one of embodiments 15-19, wherein l₁ and l₂ are each at least about 256 msec.
21. A system according to one of embodiments 15-20, wherein the quality metric is determined from a minimum-to-maximum amplitude (“minmax”), a normalized energy for 120 msec interval (“nE”), a variance of a derivative calculated for the segment (“nVD”), and a number of threshold crossings (“THC”) for window n.
22. A system according to embodiment 21, wherein
MinMax(n)=max(x[n])−min(x[n]), where x[n] is the amplitude of the filtered seismocardiogram waveform in window n;

$$nE ( n ) = ( {\sum }_{n = 1}^{0.12 * Fs} {x [ n ]}^{2} - {\sum }_{n = 0.12 * Fs}^{N 1} {x [ n ]}^{2} ) / ( {\sum }_{n = 1}^{0.12 * Fs} {x [ n ]}^{2} + {\sum }_{n = N 2}^{Nb} {x [ n ]}^{2} ) ; \\ nVD ( n ) = MinMax ( n ) / ( 1 + ( {\sum }_{n = 2}^{Nb} {( x [ n ] - x [ n - 1 ] )}^{2} ) / ( Nb - 1 ) ) ; \\ and \\ THC ( n ) = {\sum }_{n = 1}^{Nb - 1} f ( ( x [ n ] - Th ) * ( x [ n + 1 ] - Th ) ) \qquad ( 5 )$$

where f(s)=1 if s<0 otherwise f(s)=0, Th=r*Max(x[n]), x[n]=1,2, . . . ,Nwin, 0<r<1
   23. A system according to one of embodiments 15-22, wherein the template is an average seismocardiogram waveform window calculated from at least 20 windows meeting a quality metric.
   24. A system according to one of embodiments 15-22, wherein the template is an average seismocardiogram waveform window calculated from at least 30 windows meeting a quality metric.
   25. A system according to one of embodiments 15-22, wherein the template is an average seismocardiogram waveform window calculated from at least 40 windows meeting a quality metric.
   26. A system according to one of embodiments 15-22, wherein the template is an average seismocardiogram waveform window calculated from at least 60 windows meeting a quality metric.
   27. A system according to one of embodiments 15-26, wherein executing the code further performs the following steps

for each aortic valve opening m and QRS complex m, calculating a preejection period (PEP) m as the time difference between the onset of QRS complex m and occurrence of aortic valve opening m; and

displaying each PEPm on a display device.

28. A system according to embodiment 27, wherein the system further comprises a photoplethysmogram sensor configured to position externally on the hand of the individual and generate a time-dependent photoplethysmogram waveform, and wherein the processing component is operably connected the photoplethysmogram sensor to receive the time-dependent photoplethysmogram waveform, and wherein executing the code further performs the following steps

for each aortic valve opening m and QRS complex m, calculating a pulse transit time (PTT) m using PEP m, and a continuous noninvasive blood pressure (cNIBP) value m using PTT m; and

displaying the cNIBP value m on the display device.

While the invention has been described and exemplified in sufficient detail for those skilled in this art to make and use it, various alternatives, modifications, and improvements should be apparent without departing from the spirit and scope of the invention. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. A method of monitoring fiduciary features in the cardiac cycle of an individual, comprising:
generating a time-dependent seismocardiogram waveform using a vibration sensor located on the thorax of the individual;
generating a corresponding time-dependent ECG waveform using an ECG sensor located on the individual; and
receiving the time-dependent seismocardiogram waveform and the time-dependent ECG waveform on a processing component and executing code on the processing component, wherein executing the code performs the following steps to process the time-dependent seismocardiogram waveform and the time-dependent ECG waveform:
filtering the time-dependent seismocardiogram waveform to a frequency band between 0 Hz and 100 Hz to create a filtered seismocardiogram waveform;
creating a template, wherein the template is an average seismocardiogram waveform window calculated from at least 10 windows meeting a quality metric, by
(i) for each QRS complex n identified in the time-dependent ECG waveform, segmenting the filtered seismocardiogram waveform in a window n, with each window being l₁ msec in length,
(ii) determining a quality metric for window n for potential inclusion in the template,
(iii) including window n in the template if the quality metric is acceptable, and
(iv) repeating (i)-(iii) until at least 30 windows are included in the template;
identifying a fiducial point in the template indicative of aortic valve opening; and
for each subsequent QRS complex m in the filtered seismocardiogram waveform, identifying an aortic valve opening m corresponding to the QRS complex m in the filtered seismocardiogram waveform by segmenting the filtered seismocardiogram waveform in a window m with each window m being l₂ msec in length, and comparing window m to the template using a fitness function to identify a fiducial point in window m that matches the fiducial point in the template.

2. A method according to claim 1, wherein each subsequent QRS complex m in the filtered seismocardiogram waveform is used to update the template according to steps (i)-(iv).

3. A method according to claim 2, wherein the vibration sensor is selected from the group consisting of an accelerometer, a gyroscope, a laser Doppler vibrometer, a microwave Doppler vibrometer, and an airborne ultrasound surface motion camera.

4. A method according to claim 3, wherein the time-dependent seismocardiogram waveform is recorded on a dorsoventral axis.

5. A method according to claim 4, wherein the frequency band is between about 6 Hz and about 60 Hz.

6. A method according to claim 1-5, wherein l₁ and l₂ are each at least about 256 msec.

7. A method according to claim 1, wherein the quality metric is determined from a minimum-to-maximum amplitude (“minmax”), a normalized energy for 120 msec interval (“nE”), a variance of a derivative calculated for the segment (“nVD”), and a number of threshold crossings (“THC”) for window n.

8. A method according to claim 7, wherein
MinMax(n)=max(x[n])−min(x[n]), where x[n] is the amplitude of the filtered seismocardiogram waveform in window n;
$$nE ( n ) = ( {\sum }_{n = 1}^{0.12 * Fs} {x [ n ]}^{2} - {\sum }_{n = 0.12 * Fs}^{N 1} {x [ n ]}^{2} ) / ( {\sum }_{n = 1}^{0.12 * Fs} {x [ n ]}^{2} + {\sum }_{n = N 2}^{Nb} {x [ n ]}^{2} ) ; \\ nVD ( n ) = MinMax ( n ) / ( 1 + ( {\sum }_{n = 2}^{Nb} {( x [ n ] - x [ n - 1 ] )}^{2} ) / ( Nb - 1 ) ) ; \\ and \\ THC ( n ) = {\sum }_{n = 1}^{Nb - 1} f ( ( x [ n ] - Th ) * ( x [ n + 1 ] - Th ) ) \qquad ( 5 )$$
where f(s)=1 if s 21 0 otherwise f(s)=0, Th=r*Max(x[n]), x[n]=1,2, . . . ,Nwin, 0<r<1

9. A method according to claim 8, wherein the template is an average seismocardiogram waveform window calculated from at least 20 windows meeting a quality metric.

10. A method according to claim 8, wherein the template is an average seismocardiogram waveform window calculated from at least 30 windows meeting a quality metric.

11. A method according to claim 8, wherein the template is an average seismocardiogram waveform window calculated from at least 40 windows meeting a quality metric.

12. A method according to claim 8, wherein the template is an average seismocardiogram waveform window calculated from at least 60 windows meeting a quality metric.

13. A method according to claim 1, wherein executing the code further performs the following steps
for each aortic valve opening m and QRS complex m, calculating a preejection period (PEP) m as the time difference between the onset of QRS complex m and occurrence of aortic valve opening m; and
displaying each PEPm on a display device.

14. A method according to claim 13, wherein executing the code further performs the following steps
for each aortic valve opening m and QRS complex m, calculating a pulse transit time (PTT) m using PEP m, and a continuous noninvasive blood pressure (cNIBP) value m using PTT m; and
displaying the cNIBP value m on the display device.

15. A system for monitoring fiduciary features in the cardiac cycle of an individual, comprising:
a vibration sensor configured to position externally on the thorax of the individual and generate a time-dependent seismocardiogram waveform;
an ECG sensor configured to position externally on the individual and generate a time-dependent ECG waveform; and
a processing component comprising a microprocessor and a non-volatile memory operably connected to the microprocessor, wherein the processing component is operably connected the vibration sensor and the ECG sensor to receive the time-dependent seismocardiogram waveform and the time-dependent ECG waveform and is configured to execute code stored on the processing component, wherein executing the code performs the following processing steps on the time-dependent seismocardiogram waveform and the time-dependent ECG waveform
filtering the time-dependent seismocardiogram waveform to a frequency band between 0 Hz and 100 Hz to create a filtered seismocardiogram waveform;
creating a template, wherein the template is an average seismocardiogram waveform window calculated from at least 10 windows meeting a quality metric, by
(i) for each QRS complex n identified in the time-dependent ECG waveform, segmenting the filtered seismocardiogram waveform in a window n, with each window being l₁ msec in length, with l₁ being at least about 256 msec,
(ii) determining a quality metric for window n for potential inclusion in the template,
(iii) including window n in the template if the quality metric is acceptable,
(iv) repeating (i)-(iii) until at least 30 windows are included in the template, and
identifying a fiducial point in the template indicative of aortic valve opening; and
for each subsequent QRS complex m in the filtered seismocardiogram waveform, identifying an aortic valve opening m corresponding to the QRS complex m in the filtered seismocardiogram waveform by segmenting the filtered seismocardiogram waveform in a window m with each window m being l₂ msec in length, with l₂ being at least about 256 msec, and comparing window m to the template using a fitness function and identifying a fiducial point in window m that matches the fiducial point in the template.

16. A system according to claim 15, wherein each subsequent QRS complex m in the filtered seismocardiogram waveform is used to update the template according to steps (i)-(iv).

17. A system according to claim 16, wherein the vibration sensor is selected from the group consisting of an accelerometer, a gyroscope, a laser Doppler vibrometer, a microwave Doppler vibrometer, and an airborne ultrasound surface motion camera.

18. A system according to claim 17, wherein the time-dependent seismocardiogram waveform is recorded on a dorsoventral axis.

19. A system according to claim 18, wherein the frequency band is between about 6 Hz and about 60 Hz.

20. A method according to claim 19, wherein l₁ and l₂ are each at least about 256 msec.

21. A system according to claim 20, wherein the quality metric is determined from a minimum-to-maximum amplitude (“minmax”), a normalized energy for 120 msec interval (“nE”), a variance of a derivative calculated for the segment (“nVD”), and a number of threshold crossings (“THC”) for window n.

22. A system according to claim 21, wherein
MinMax(n)=max(x[n])−min(x[n]), where x[n] is the amplitude of the filtered seismocardiogram waveform in window n;
$$nE ( n ) = ( {\sum }_{n = 1}^{0.12 * Fs} {x [ n ]}^{2} - {\sum }_{n = 0.12 * Fs}^{N 1} {x [ n ]}^{2} ) / ( {\sum }_{n = 1}^{0.12 * Fs} {x [ n ]}^{2} + {\sum }_{n = N 2}^{Nb} {x [ n ]}^{2} ) ; \\ nVD ( n ) = MinMax ( n ) / ( 1 + ( {\sum }_{n = 2}^{Nb} {( x [ n ] - x [ n - 1 ] )}^{2} ) / ( Nb - 1 ) ) ; \\ and \\ THC ( n ) = {\sum }_{n = 1}^{Nb - 1} f ( ( x [ n ] - Th ) * ( x [ n + 1 ] - Th ) ) \qquad ( 5 )$$
where f(s)=1 if s<0 otherwise f(s)=0, Th=r*Max(x[n]), x[n]=1,2, . . . ,Nwin, 0<r<1

23. A system according to claim 22, wherein the template is an average seismocardiogram waveform window calculated from at least 20 windows meeting a quality metric.

24. A system according to claim 22, wherein the template is an average seismocardiogram waveform window calculated from at least 30 windows meeting a quality metric.

25. A system according to claim 22, wherein the template is an average seismocardiogram waveform window calculated from at least 40 windows meeting a quality metric.

26. A system according to claim 22, wherein the template is an average seismocardiogram waveform window calculated from at least 60 windows meeting a quality metric.

27. A system according to claim 15, wherein executing the code further performs the following steps
for each aortic valve opening m and QRS complex m, calculating a preejection period (PEP) m as the time difference between the onset of QRS complex m and occurrence of aortic valve opening m; and
displaying each PEPm on a display device.

28. A system according to claim 27, wherein the system further comprises a photoplethysmogram sensor configured to position externally on the hand of the individual and generate a time-dependent photoplethysmogram waveform, and wherein the processing component is operably connected the photoplethysmogram sensor to receive the time-dependent photoplethysmogram waveform, and wherein executing the code further performs the following steps for each aortic valve opening m and QRS complex m, calculating a pulse transit time (PTT) m using PEP m, and a continuous noninvasive blood pressure (cNIBP) value m using PTT m; and
displaying the cNIBP value m on the display device.
